(19) United States
(12) Patent Application Publication
(10) Pub. No.: US 2005/0019263 A1
(43) Pub. Date: Jan. 27, 2005

(54) Conjugate for differentiating between healthy and unhealthy tissue

(76) Inventors: Hannsjorg Sinn, Wiesloch (DE); Hans-Hermann Schrenk, Zeiskamm (DE); Andreas Wunder, Eppelheim (DE); Gerd Stehle, Heidelberg (DE)

Correspondence Address: DORSEY & WHITNEY LLP;INTELLECTUAL PROPERTY DEPARTMENT, 4 EMBARCADERO CENTER, SUITE 3400, SAN FRANCISCO, CA, 94111, US

(21) Appl. No.: 10/917,907

(22) Filed: Aug. 13, 2004

Related U.S. Application Data
(63) Continuation of application No. 09/463,474, filed on Aug. 4, 2000, now abandoned.

(30) Foreign Application Priority Data
Jul. 23, 1997 (DE) 197 51 741.3

(51) Int. Cl. A61K 49/00; G01N 33/53

(52) U.S. Cl. 424/9.6; 435/7.1; 530/350; 530/409

(57) ABSTRACT

The present invention relates to conjugates for differentiating between healthy and unhealthy tissue, methods of producing such conjugates as well as their use.

This is a national phase filing of the Application No. PCT/DE98/02 102, which was filed with the Patent Corporation Treaty on Jul. 22, 1998, and is entitled to priority of German Patent Application 197 31 741.3 filed Jul. 23, 1997.

### I. FIELD OF THE INVENTION

The present invention relates to conjugates for differentiating between healthy and unhealthy tissue, methods of producing such conjugates as well as their use.

### II. BACKGROUND OF THE INVENTION

For the treatment of unhealthy tissue, e.g. of tumors, the removal thereof is often an essential measure. For this purpose, it is necessary for the operating surgeon to recognize accurately where unhealthy tissue ends and where healthy tissue starts. However, this is often impossible. As a result, offshoots of the unhealthy tissue are overlooked, which are then the basis for another formation of the unhealthy tissue.

Therefore, it is the object of the present invention to provide a product by means of which a differentiation can be made between unhealthy and healthy tissue.

### III. SUMMARY OF THE INVENTION

The present invention relates to conjugates for differentiating between healthy and unhealthy tissue, methods of producing such conjugates as well as their use.

### IV. BRIEF DESCRIPTION OF THE DRAWINGS

The invention is described in more detail by the below FIGURES.

FIG. 1 shows the production of a conjugate from acridine-9-carboxylic acid and human serum albumin.

FIG. 2 shows the production of a conjugate from coumarin 343 and human serum albumin.

FIGURE shows the production of a conjugate from tetrasulfophenylporphin and human serum albumin.

### V. DETAILED DESCRIPTION OF THE INVENTION

It is the object of the present invention to provide a product by means of which a differentiation can be made between unhealthy and healthy tissue. According to the invention this is achieved by the subject matters defined in the claims.

Thus, the subject matter of the present invention relates to a conjugate, comprising a fluorescent compound and a carrier, wherein the compound and the carrier are connected via an acidic ester or acidic amide bond or enane bridge (schiff base) and the compound has an excitation wavelength of 630 nm or more and/or 450 nm or less.

The expression “carrier” comprises compounds of any kind which are suited for the enrichment of the conjugate in a certain tissue, e.g. a tumor, a focus of inflammation or in superficial, relatively small vessels, such as neovascularizations in the area of the cornea. Examples of such carriers are proteins and polyether. For forming the acidic ester or acidic amide bond with the fluorescent compound, the carrier may include hydroxyl or amino groups.

The proteins are preferably not considered foreign to the body. They may be present in native form. In the native form, the proteins have no intermolecular and/or intramolecular cross-linking. The proteins favorably have a molecular weight of up to 100,000 Dalton, particularly 30,000 to 100,000 Dalton. Furthermore, it is favorable for the proteins to be human proteins. Examples of the proteins are albumin, fibrinogen, transferrin, immunoglobulins and lipoproteins, human serum albumin (HSA) being preferred. It is also possible to use fragments of the above proteins. In addition, the sequence of the proteins and the fragments thereof, respectively, may comprise modifications of one or several amino acids over known sequences of the proteins and fragments thereof, respectively.

Examples of the polyethers are polyethylene glycols, particularly those having a molecular weight of 100 to 20,000 Dalton. The polyethylene glycols are preferably esterified or etherified with a C₁-C₁₂ alkyl group, particularly with a methyl group, on the terminal hydroxyl group.

A conjugate according to the invention may have one or several, particularly 2 to 4, of the above carriers. If several carriers are present, they may be equal or differ from one another. If several polyethers are present, they will favorably be selected such that the molecular weight of all polyethers is about 20,000 Dalton or more.

The expression “fluorescent compound” comprises compounds of any kind which can be induced to display fluorescence. These compounds can also be photoactive. The compound is connected with the carrier via an acidic ester or acidic amide bond or enane bridge. For the formation thereof, the fluorescent compound may comprise an acid group, e.g. a carboxylic, sulfonic, phosphonic or arsonic acid group, a hydroxyl group, an amino group or an aldehyde group. Several of these groups may be present, which may be equal or differ from one another. The fluorescent compound is excited at a wavelength of 640 nm or more, preferably 630 to 850 nm, and particularly preferably 650 to 850 nm, and/or at a wavelength of 450 nm or less, preferably 320 to 450 nm. These wavelengths refer to the excitation wavelengths which the fluorescent compound has in the conjugate according to the invention; in a free form, their excitation wavelength may differ therefrom. Representatives of these compounds are porphyrins such as tetrasulfophenyl porphyrin (TSPP; excitation wavelength 650 nm when bound to HSA), chlorins, bacteriochlorins, chlorophylls, phthalocyanines, wherein these compounds may include metal ions as central atom. Furthermore, representatives of the fluorescent compound are carboxy cinnamic acid, carboxy fluorescein, acridine carboxylic acid, such as acridine-9-carboxylic acid, coumaric acid, such as coumarin 343, coumarin-3-carboxylic acid, and hydroxy coumarin acetic acid (excitation wavelength 365 nm when bound to HSA), and indocyanine green (excitation wavelength 805 nm when bound to HSA) as well as derivatives of the above compounds.

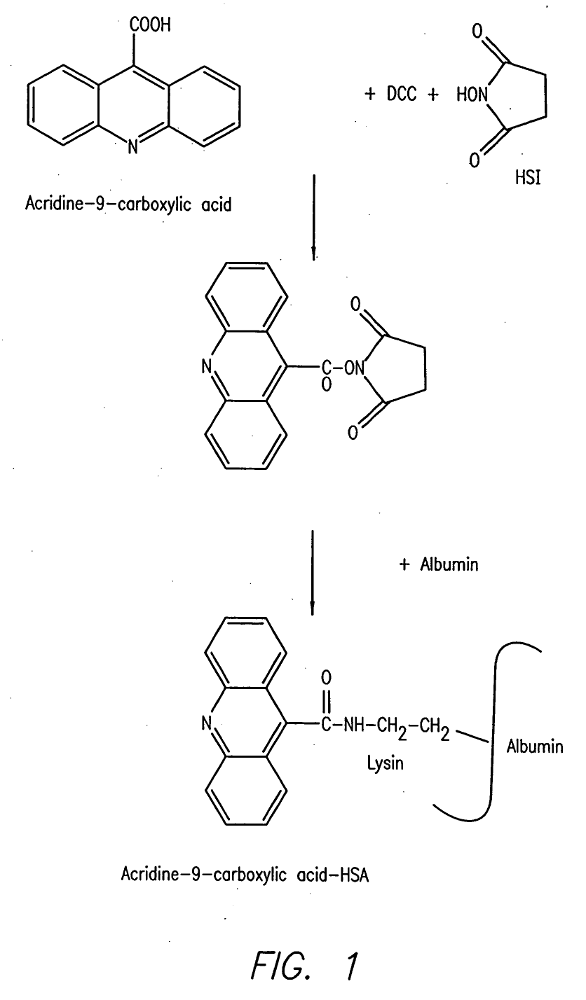

One or several fluorescent compounds can be present in the conjugate according to the invention. If several are present, they may be the same or differ from one another. Particularly preferred conjugates according to the invention are shown in FIGS. 1 to 3.

Conjugates according to the invention can be produced by covalently bonding the fluorescent compound with the carrier thereby forming an acidic ester or acidic amide bond. A person skilled in the art is familiar with methods suitable for this purpose as well as necessary materials.

If the fluorescent compound includes an acid group, the conjugates can be produced by reacting this compound with carbodiimide and hydroxy succinimide into reactive succinimidyl esters and the latter can then be converted with the carrier. In the case of conjugates having several fluorescent compounds, the succinimidyl esters can be produced jointly or separately.

The fluorescent compound is reacted with carbodiimide and hydroxy succinimide in a polar aprotic solvent, preferably dimethyl formamide or dimethyl sulfoxide (DMSO). The molar ratio of fluorescent compound:carbodiimide:hydroxy succinimide is about 1:1.5-3:5-10. The resulting succinimidyl ester is then reacted in an aqueous buffer solution, preferably NaHCO₃, with the carrier, such as albumin. The carrier concentration is about 10 to 70 mg/ml. The thus activated acid group can then react with OH and NH groups of the carrier thereby forming acidic amide or acidic ester bonds, conjugates according to the invention being obtained. The conjugates can be purified several times, e.g. by ultrafiltration, and finally be sterile filtered. Thereafter, they are ready for application.

Conjugates according to the invention distinguish themselves by a prolonged half life in the organism. In addition, conjugates according to the invention accumulate in unhealthy tissue, particularly in tumoral tissue, in foci of inflammation and in superficial relatively small vessels, e.g. of neovascularizations in the area of the cornea. The fluorescent compound is excited or activated by light, so that unhealthy tissue can be made visible, whereas healthy tissue in which the conjugates according to the invention to not accumulate is not made visible. Furthermore, there is no disturbance caused by the inherent fluorescence of blood or tissue, e.g. the liver, so that the optical impression is not falsified. In addition, conjugates according to the invention, in which the fluorescent compound can be excited at 630 nm or more, have a great penetration depth.

The below examples explain the invention in more detail. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

### VI. EXAMPLES

### A. Example 1

### Production of a conjugate according to the invention from acridine-9-carboxylic acid and human serum albumin

The structure and the production of the conjugate are shown in FIG. 1.

20 mg of acridine-9-carboxylic acid hydrate (A9CA) were dissolved in 2 ml DMSO and about 100 mg of N-hydroxysuccinimide (HSI) in a molar ratio of about 10/1 as well as 30 mg N,N′-di-cyclohexyl carbodiimide (DCC) in a molar ratio or about 1.5/a were added. After about 6 hours, the formation of the hydroxysuccinimidyl ester is concluded. Following the separation of the dicyclohexyl urea (DCHU) through a solvent-resistant filter (0.2 μm), the ester is slowly added to a solution of 2 g of human serum albumin (HSA) which is dissolved in 10 ml of original solution, 10 ml of 0.34 M NaHCO₃ and 10 ml of methoxypolyethylene glycol (MPEG). The slight clouding resulting upon the addition disappears again after a short time. A slightly yellowish solution of a conjugate from A9CA and HSA results. The accompanying substances undesired in the finished preparation, such as excess DCC, HSI, unbound A9CA, DMSO and MPEG, are separated by means of ultrafiltration (exclusion limit 10 kD) comprising at least 4 wash steps.

### B. Example 2

### Production of a conjugate according to the invention from coumarin 343 and human serum albumin

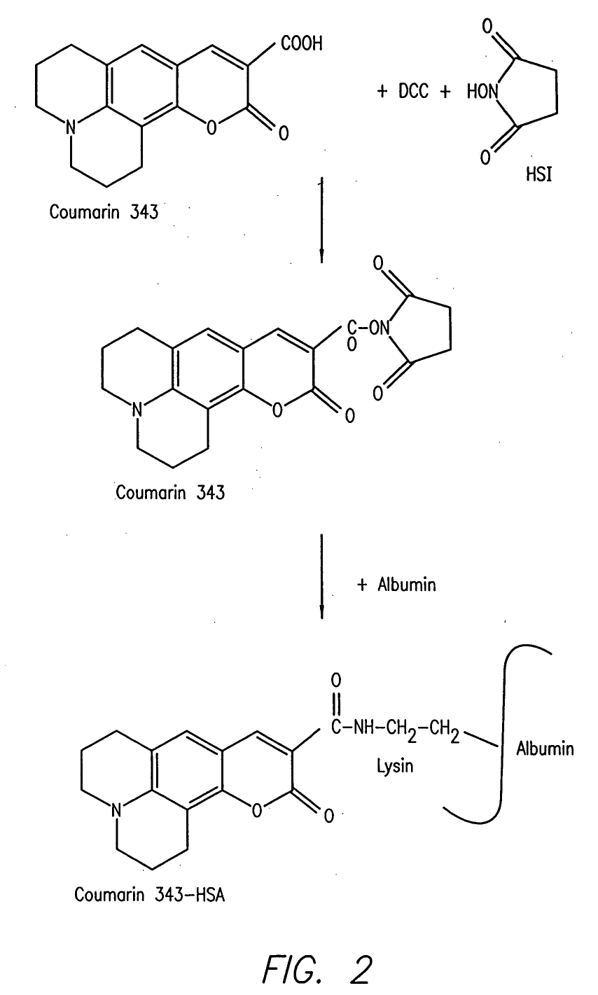

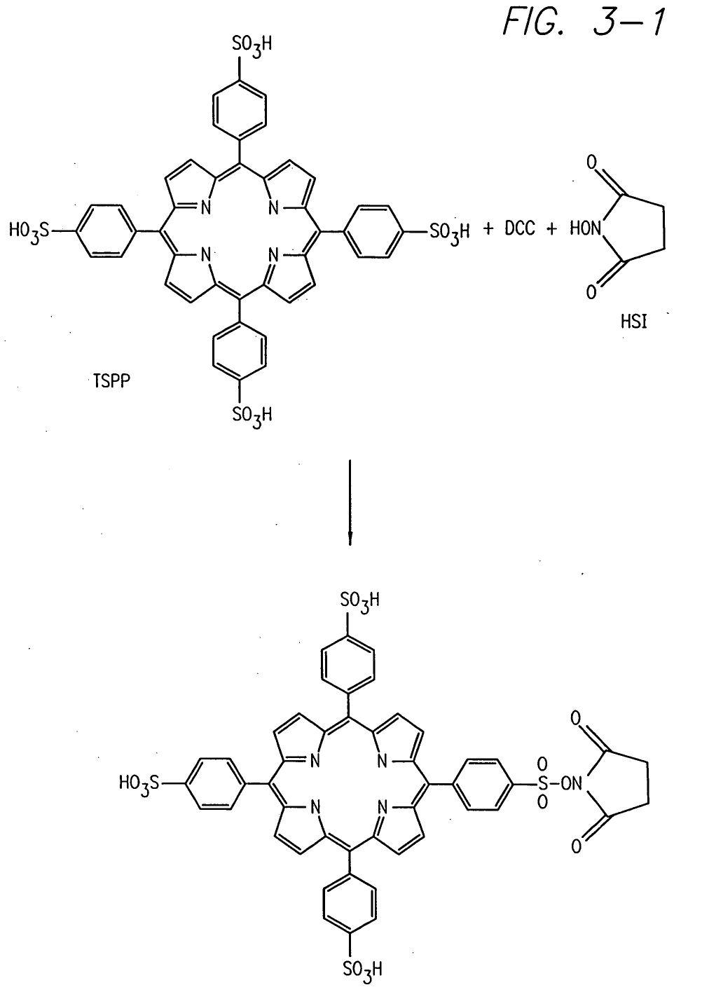

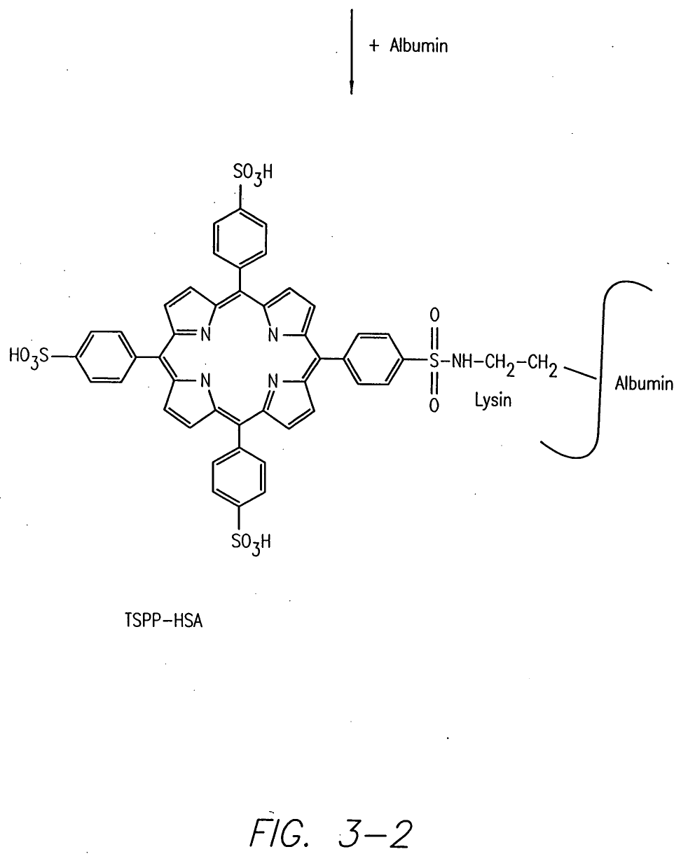

The structure and the production of the conjugate are shown in FIG. 2.

20 mg of coumarin 343 (C343=10-carboxy-2,3,6,7-tetrahydro-1H,5H,11H-[1]benzopyranone[6,7,8,ij]-quinolizine-11-one) were dissolved in 2 ml DMSO. For this purpose, about 100 mg HSI in a molar ratio of 10/1 and 30 mg DCC in a molar ratio of about 1.5/1 were added. The ester was isolated as described in Example 1 and reacted with HSA, an intensely yellow solution of a conjugate from C343 and HSA being obtained. Undesired accompanying substances are separated as described in Example 1.

### C. Example 3

### Production of a conjugate according to the invention from tetra-(4-sulfophenyl)porphin and human serum albumin

The structure of the conjugate and its production are shown in FIG. 3.

Tetra-(4-sulfophenyl)porphin (TSPP) was dissolved in a concentration of 10 mg/ml in DMSO. Three times the molar amount of DCC and five times the molar amount of HSI were added to the clear dark green solution. After a reaction period of about 3 to 4 hours, the conversion into TSPP succinimidyl ester (TSPP-SE) is concluded, the resulting di-cyclohexyl urea being separated in the form of fine grains. The analytical control is carried out by means of thin-layer chromatography.

Human serum albumin (HSA, 4 g, i.e. 2 ampoules of 2 g in 10 ml each) were diluted with 2×10 ml of 0.17 M NaHCO₃ and 20 ml of methoxypolyethylene glycol₃₅₀ and charged to a 100 ml Erlenmeyer flask. The above TSPP-SE solution in DMSO was slowly added to this HSA solution with constant stirring, the initially clear solution becoming cloudy because of non-reacted DCC which is insoluble in aqueous solution. Having concluded the addition of TSPP-SE, the reaction mixture was stirred at room temperature for 30 minutes so as to complete the reaction. Thereafter, the turbid matter was separated via a sterile filter unit (Millipore, Stericup—GV, 0.22 μm Low Binding Duropore Membrane) and the low-molecular water-soluble components (DMSO, HSI and unbound TSPP) were separated by ultrafiltration via a membrane having 30 kD exclusion limit (Amicon YM 30). A conjugate according to the invention was obtained from TSPP and HSA. The linkage yield of TSPP to HSA was 85 to 90%.

The analytical purity was controlled by means of HPLC under the following conditions:

| | |
|---|---|
| Precolumn: | Zorbax Diol (50 × 4 mm) |
| Column 1: | Zorbax GF 450 |
| Column 2: | Zorbax GF 450 |
| Running agent: | 0.2 M Na citrate, pH 7.5 |
| Flow: | 1 ml/min |
| Detector 1: | 280 nm (for the protein) |
| Detector 2: | 420 m (for TSPP) |

All references cited within the body of the instant specification are hereby incorporated by reference in their entirety.

1. A conjugate, comprising a fluorescent compound and a carrier, wherein the compound and the carrier are connected via an acidic ester or acidic amide bond or an enane bridge, the compound in the conjugate has an excitation wavelength of 630 nm or more and/or 450 nm or less.

2. The conjugate according to claim 1, characterized in that the carrier is a protein.

3. The conjugate according to claim 2, characterized in that the protein is a native protein which is not regarded as foreign to the body.

4. The conjugate according to claims 3, characterized in that protein is human serum albumin.

5. The conjugate according to claim 1, characterized in that the carrier is a polyether.

6. The conjugate according to claim 5, characterized in that the polyether is a polyethylene glycol.

7. The conjugate according to any one of claims 1 to 6, characterized in that several carriers are present.

8. The conjugate according to any one of claims 1 to 7, characterized in that the fluorescent compound comprises an acid group, hydroxyl group, amino group or aldehyde group.

9. The conjugate according to any one of claims 1 to 8, characterized in that the excitation wavelength is 630 to 850 nm.

10. The conjugate according to any one of claims 1 to 9, characterized in that the excitation wavelength is 320 to 450 nm.

11. The conjugate according to any one of claims 1 to 10, characterized in that the fluorescent compound is derived from porphyrin, chlorin, bacteriochlorin, chlorophyll, phthalocyanine, carboxy cinnamic acid, carboxyfluorescein, acridic acid, coumaric acid or indocyanine green as well as the derivatives thereof.

12. The conjugate according to any one of claims 1 to 11, characterized in that several fluorescent compounds are present.

13. A method of producing a conjugate according to any one of claims 1 to 12, characterized in that the fluorescent compound and the carrier are covalently bonded thereby forming an acidic ester or acidic amide bond.

14. Use of a conjugate according to any one of claims 1 to 12 for differentiating between healthy and unhealthy tissue.
